(19) United States
(12) Patent Application Publication
(10) Pub. No.: US 2026/0311881 A1
(43) Pub. Date: Oct. 8, 2026

(54) Non-Contact Intelligent Health Monitoring Method and System Using DrAI Screen Technology

(71) Applicant: FURONG BEI, SAN GABRIEL, CA (US)

(72) Inventor: FURONG BEI, SAN GABRIEL, CA (US)

(21) Appl. No.: 19/069,264

(22) Filed: Mar. 4, 2025

(51) Int. Cl. G16H 50/30; G06V 10/25; G06V 10/62; G06V 10/80; G06V 10/82; G06V 40/16; G06V 40/20; G10L 25/66; G10L 25/90; G16H 40/67; G16H 50/20

(52) U.S. Cl. CPC G16H 50/30; G06V 10/25; G06V 10/62; G06V 10/806; G06V 10/82; G06V 40/171; G06V 40/174; G06V 40/20; G10L 25/66; G10L 25/90; G16H 40/67; G16H 50/20

(57) ABSTRACT

A non-contact intelligent health monitoring method and system uses a smartphone or device screen associated with a camera, microphone, environmental sensing interface, processor, and memory. Facial image data and respiratory movement data are captured without physical contact, voice data are recorded, and particulate matter information is received. A signal-quality gate evaluates visual, acoustic, and environmental adequacy. Facial features including complexion, expression, micro-expression, or eye-movement information are extracted; respiratory features are extracted using Eulerian Video Magnification or video motion processing; acoustic features are extracted from voice data; and environmental features are generated from particulate information. A DrAI analysis module fuses the extracted features to output a preliminary non-diagnostic health assessment or real-time early warning alert through the smart device, while maintaining a screen-facing, quality-controlled, multi-modal workflow and indicating that outputs are preliminary and do not replace professional medical diagnosis or care.

### TECHNICAL FIELD

The disclosure relates to screen-based non-contact health monitoring, health-risk observation, signal processing, video-based respiratory assessment, voice-based health analysis, environmental particulate sensing, and real-time medical early-warning support. More particularly, the disclosure relates to a smartphone or device screen technology that obtains facial, respiratory, vocal, and environmental information without requiring a body-contact sensor and processes the information through a DrAI analysis module to provide a preliminary health assessment or early warning output.

The disclosed technology is implemented through device-screen-associated acquisition and analysis components rather than through a generic mental process. The components include a screen-associated camera, an integrated microphone, one or more environmental sensing interfaces, one or more processors, memory storing instructions, a display or alert output interface, feature extraction routines, signal-quality operations, and DrAI multi-modal fusion operations. These structures cooperate to form a concrete monitoring workflow capable of operating during an ordinary screen-facing session.

### BACKGROUND

Smartphones and smart devices are widely used and commonly include cameras, microphones, processors, memories, sensors, network interfaces, and screens. A user may be in front of such a device many times each day, yet conventional screen use does not normally convert that screen interaction into a structured health observation session. A device screen therefore presents an opportunity for convenient health monitoring that does not require a separate wearable, a dedicated probe, or a direct physical contact measurement.

Traditional health monitoring systems may require direct physical contact with a user, attachment of electrodes, use of external medical sensors, manual entry, or separate environmental monitoring devices. Such approaches can be valuable, but they may be inconvenient, forgotten, incorrectly placed, unavailable during a remote event, or unsuitable for frequent preliminary checks. A screen-associated system can reduce those barriers by using a camera, microphone, and environmental interface already available in or near a smart device.

A conventional video call may allow a remote observer to view a user, and a conventional camera may record video, but such arrangements do not necessarily extract facial features, respiratory movement features, acoustic voice features, and environmental particulate features as a synchronized screen-based health monitoring workflow. Similarly, a conventional environmental monitor may report particulate matter, and a conventional voice application may capture speech, but those systems do not form the integrated DrAI analysis loop described herein.

There is therefore a need for a non-contact intelligent health monitoring method and system that uses a smartphone or device screen, captures facial and respiratory video information, records voice information, receives environmental particulate information, evaluates the adequacy of the captured data, fuses the resulting feature sets, and produces a preliminary non-diagnostic health assessment or early warning alert in real time.

### SUMMARY

The invention provides a non-contact intelligent health monitoring method and system utilizing smartphone or device screens. The system captures facial image data, respiratory movement data, voice data, and environmental particulate data through screen-associated cameras, microphones, and sensors or sensor interfaces. The captured data are analyzed using DrAI algorithms to provide comprehensive real-time health monitoring and early warning without requiring additional hardware attached to the user.

In one embodiment, a screen-associated camera captures facial video frames and respiratory movement frames while a user is positioned before a smartphone or device screen. An integrated microphone records voice data. An environmental sensor or environmental sensing interface obtains particulate matter information relating to the user environment. A processor applies signal adequacy checks, extracts facial features, respiratory features, acoustic features, and environmental features, and fuses the extracted data through a DrAI analysis module to generate a preliminary health assessment and an early warning alert when appropriate.

The disclosed system is not limited to a single feature source. Facial data may include complexion, expression, micro-expression, or eye-movement information. Respiratory data may include motion-derived rate, depth, rhythm, or confidence information obtained using Eulerian Video Magnification or video motion processing. Voice data may provide acoustic features such as pitch, energy, spectral, timing, pause, or speech-rate information. Environmental data may provide particulate matter information associated with the monitoring session.

The system includes a signal-quality gate that determines whether visual, acoustic, and environmental inputs are adequate before a final preliminary output is produced. If a feature source is insufficient, the system may request a repeat capture, reduce influence of the feature, mask the feature, exclude the feature, or generate a limited output. This quality-control structure provides practical reliability and helps prevent over-statement of a result derived from poor-quality input.

The output is a preliminary non-diagnostic health assessment or real-time early warning alert. The output may be displayed on the smart device screen or delivered through an authorized device output path. The output is not a definitive medical diagnosis, clinical measurement, emergency medical service, continuous clinical monitoring system, or substitute for professional medical care.

### No-New-Matter Implementation Principle

The description herein is organized to explain the originally disclosed screen-based DrAI health monitoring system in a detailed and compliant manner. The disclosure is directed to facial, respiratory, vocal, and environmental particulate analysis through smartphone or device screen technology and does not depend on unrelated financial, domain registry, blockchain, rescue-center, resource-center, fall-detection, or clinical treatment systems.

The terminology, examples, and implementation details are intended to explain how the originally disclosed screen-based monitoring system can be implemented by a person of ordinary skill using a camera, microphone, environmental sensing interface, processor, memory, feature extraction logic, DrAI analysis module, and real-time alert output. The claims should be understood as directed to the recited technical structures and operations, not to unrelated commercial or institutional ecosystems.

### Definitions and Naming Non-Limitation

A screen-based device, device screen, or screen-associated terminal may include a smartphone, tablet, laptop, desktop computer, smart display, or other smart device having a user-facing screen and associated camera, microphone, processor, memory, and communication interface. The screen serves as a user interface for positioning, monitoring, and alert output. The screen is not merely a passive display; it cooperates with acquisition and analysis components to form the monitoring workflow.

Non-contact monitoring means that the disclosed system can obtain facial image data, respiratory movement data, voice data, and environmental particulate data without requiring a sensor to be attached to the body of the user. The system may use the ordinary viewing relationship between a user and a device screen to capture relevant data while improving convenience and user experience.

A DrAI analysis module refers to one or more processors executing artificial intelligence, deep learning, machine learning, statistical analysis, signal processing, or rules-based processing configured to analyze facial, respiratory, vocal, and environmental information. The DrAI analysis module may be implemented locally, remotely, or in a distributed manner, and may operate with feature extraction logic, signal-quality logic, and an alert-generation rule set.

Terms such as DrAI, Doctor AI, DoctorAIR, DrAIR, DRAIR, DoctorAIRS, AIR Screen, screen-based health monitoring, or similar names may be used as product names, service names, module names, platform names, implementation names, or descriptive labels for embodiments of the disclosed screen-based non-contact health monitoring system. Such names are exemplary and do not limit the scope of the invention to a particular trademark, software title, service provider, deployment platform, or branding convention.

Unless expressly recited in a claim as a required limitation, the invention is defined by the recited technical structures, data acquisition operations, feature extraction operations, respiratory video processing operations, vocal acoustic processing operations, environmental particulate sensing operations, multi-modal analysis operations, signal-quality operations, and real-time preliminary alert or assessment operations, and not by any particular name used to identify an embodiment.

### System Architecture Overview

In an embodiment, the system includes a screen device, an image sensor, a microphone, an environmental interface, feature extraction modules, a signal-quality gate, a DrAI fusion engine, and a preliminary health-risk output. The architecture is configured so that screen-associated input is converted into structured feature data and then into a real-time assessment or early warning output. FIG. 1 schematically illustrates this closed technical loop.

The image sensor is configured to capture facial images and respiratory movement data. The microphone records voice data. The environmental interface receives particulate matter information. The processor extracts facial, respiratory, vocal, and environmental features and applies the DrAI module to produce a health assessment and alert. The device screen may provide capture instructions, status indicators, repeat-capture prompts, preliminary output messages, and non-diagnostic notices.

The architecture is designed to be practical on ordinary smart devices. The system may use a front-facing camera, built-in microphone, processor, memory, and display, while receiving environmental particulate information from an embedded sensor, connected sensor, or associated environmental sensing interface. No body-contact sensor is required for the claimed core operation, although the system does not exclude optional additional sensors outside the claimed minimum configuration.

Because the system uses heterogeneous signal sources, the architecture includes quality and synchronization operations. The visual path may be affected by lighting, frame rate, occlusion, motion blur, and user position. The acoustic path may be affected by noise, clipping, silence, or insufficient speech. The environmental path may be affected by unavailable, invalid, or stale particulate information. The signal-quality gate provides a technical mechanism for handling those conditions before an output is generated.

The system may be implemented as a smartphone application, a smart-device program, a screen-associated software service, embedded device software, or a combination of local and remote software components. In each implementation, the monitoring operation remains centered on the same non-contact screen-based acquisition, feature extraction, quality gate, DrAI fusion, and preliminary alert output.

### Session Activation and Screen Prompting

A monitoring session may be initiated when the user opens a health monitoring application, responds to a device prompt, or otherwise positions the user before the screen. The screen may display a prompt instructing the user to face the camera, remain within a capture window, speak a sample phrase, breathe normally, or remain still for a respiratory capture interval. This screen prompt converts an ordinary screen-facing posture into a structured monitoring session.

The prompt may include visual alignment cues, timing indicators, progress messages, or repeat-capture instructions. For example, the screen may show whether the user's face is within the frame, whether lighting is adequate, whether the microphone is receiving a useful signal, and whether environmental data are available. These prompts are not clinical instructions; they are acquisition instructions that improve the reliability of the non-contact capture.

During the session, the system may buffer video frames, audio samples, and environmental particulate readings within a monitoring time window. The monitoring window allows the system to relate facial expression, respiratory movement, voice features, and particulate context to the same general user condition. The processor may label the data with a session time, device context, and quality state so that the DrAI module can analyze the data as a coordinated event.

The session may end when sufficient data have been acquired, when the user cancels the session, when the quality gate determines that capture cannot proceed, or when the processor has generated an assessment or repeat-capture request. The session output may then be displayed on the device screen as a preliminary non-diagnostic result, an early warning, a quality limitation, or a repeat-scan instruction.

### Screen-Associated Image Capture

The screen-associated camera captures real-time video stream data containing facial images and respiratory movement information. The camera may be embedded in the smart device or otherwise associated with the screen so that the user can naturally face the display while data are captured. The captured image stream may include facial frames, upper-body frames, neck frames, or other visible regions useful for facial and respiratory analysis.

Captured facial frames may include the face, cheeks, periocular regions, eyes, mouth region, forehead region, and other visible facial regions. Captured respiratory movement data may include subtle video movements in chest, abdominal, neck, shoulder, or facial regions when those regions are visible to the camera. The same video stream may therefore provide both facial analysis information and respiratory movement information.

The image capture operation may apply exposure control, frame selection, region tracking, temporal sampling, and quality screening. If lighting is insufficient, the face is occluded, the user is outside the capture window, the camera is unstable, or the respiratory region is not visible, the system may mark the visual signal as inadequate or request a repeat capture. The visual path is therefore not merely a passive recording; it is part of a controlled acquisition process.

The screen-associated image capture may be performed through an ordinary device camera without requiring medical-grade imaging hardware. This supports widespread use while preserving the technical nature of the invention: the camera, screen, processor, and feature extraction modules cooperate to detect facial and respiratory signals in a non-contact monitoring workflow.

### Facial Analysis Embodiments

The facial analysis subsystem may perform detecting a face region, locating landmarks, and determining whether the face is sufficiently visible and oriented toward the camera. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For face region detection, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

The facial analysis subsystem may perform extracting camera-derived facial appearance information such as color, relative intensity, temporal variation, and region-specific complexion signals. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For complexion feature extraction, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

The facial analysis subsystem may perform tracking expression-related motion patterns in mouth, cheek, eyebrow, and periocular regions over a monitoring interval. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For expression feature extraction, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

The facial analysis subsystem may perform detecting short-duration facial motion changes and using the changes as one input to a preliminary stress, fatigue, or health-risk indicator. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For micro-expression processing, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

The facial analysis subsystem may perform detecting eye opening, blink-related information, gaze stability, or visible eye-region motion as screen-visible features. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For eye movement processing, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

The facial analysis subsystem may perform adjusting or weighting facial features according to illumination, camera exposure, white balance, and shadow conditions. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For lighting normalization, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

The facial analysis subsystem may perform assigning quality or confidence values based on face visibility, pose, frame stability, and region-tracking reliability. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For facial confidence scoring, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

The facial analysis subsystem may perform organizing complexion, expression, micro-expression, and eye-movement data into a facial feature vector for DrAI fusion. This operation is tied to the original disclosure of facial recognition and analysis including complexion, expressions, micro-expressions, and eye movements. The extracted facial information is processed as a non-contact preliminary screening signal and is not presented as a definitive medical diagnosis.

For facial vector generation, the system may use the screen-associated camera to acquire multiple frames during the monitoring window and may compare the frames over time. When the facial signal is reliable, the resulting feature contributes to the DrAI assessment. When the signal is degraded by lighting, occlusion, motion, or poor positioning, the feature may be down-weighted, masked, excluded, or associated with a repeat-capture request.

### Respiratory Video Processing and EVM Embodiments

The respiration monitoring subsystem receives video information from the screen-associated camera and analyzes respiratory movement without physical contact. The system may process subtle chest, abdominal, neck, shoulder, or facial motion captured in the video to estimate respiratory characteristics. The operation is grounded in the original disclosure of Eulerian Video Magnification for non-contact respiratory assessment.

In one respiratory processing path, the processor selects a region of interest in video frames. The region may correspond to a visible chest region, neck region, lower face region, upper-body region, or other region in which breathing-related motion can appear. Region selection may be based on visibility, stability, frame coverage, and expected movement patterns during the monitoring window.

After the region of interest is selected, the processor may apply temporal filtering to emphasize changes in a respiratory frequency band and reduce unrelated variation. The temporal filter may operate on pixel intensity, landmark displacement, optical flow, phase-based motion, or another video-derived signal. This step converts raw image frames into a signal suited for respiratory feature extraction.

Eulerian Video Magnification may be applied to amplify small temporal variations corresponding to respiratory movement. The EVM processing is a concrete video-signal processing step applied to image data captured by the screen-associated camera. It improves the ability of the system to obtain non-contact respiratory assessment information from subtle physical movements visible in the video stream.

The respiratory module may produce a respiratory waveform or respiratory feature vector. The feature vector may include respiratory rate, respiratory depth, rhythm, temporal regularity, movement amplitude, confidence value, or quality flag. The features may be calculated over a selected time window and supplied to the DrAI analysis module for fusion with facial, vocal, and environmental features.

If movement artifacts, insufficient visibility, low frame rate, poor lighting, subject motion, unstable distance, or compression artifacts make the respiratory signal unreliable, the system may flag the respiratory feature as low confidence, omit the feature, or request a repeat capture. This failure-state logic preserves the non-contact nature of the system while avoiding over-reliance on poor video data.

Respiratory features may be compared with vocal features during DrAI fusion. For example, voice breathiness, cough markers, long pauses, or speech-rate changes may increase the significance of a video-derived respiratory irregularity. Conversely, when video-derived respiration is weak because of poor visibility but voice appears adequate, the system may lower confidence or request another capture rather than generating an unsupported alert.

The respiratory subsystem does not require a chest strap, spirometer, pulse oximeter, or other body-contact sensor. It extracts respiratory movement from the screen-associated video stream. This makes the system suitable for frequent preliminary checks, remote health observation, or wellness and early-warning scenarios in which contact devices are unavailable or inconvenient.

Respiratory embodiment 1 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

Respiratory embodiment 2 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

Respiratory embodiment 3 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

Respiratory embodiment 4 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

Respiratory embodiment 5 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

Respiratory embodiment 6 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

Respiratory embodiment 7 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

Respiratory embodiment 8 may use the same video stream that supports facial analysis, but may evaluate a different region, time interval, or confidence rule. The processor may determine whether the respiratory region remains sufficiently stable throughout the capture window, whether the extracted waveform is periodic enough for preliminary use, and whether the resulting respiratory indicator should be fused, downgraded, or rejected. This implementation detail supports the original non-contact respiratory assessment disclosure without adding a different invention.

### Voice and Acoustic Feature Embodiments

The voice-based analysis subsystem uses a built-in microphone or screen-associated microphone to record voice data. Acoustic features are extracted from the recorded voice data and analyzed by the DrAI module to assess emotional state, psychological stress, and indicators related to respiratory or neurological health at a preliminary, non-diagnostic level. The microphone input is therefore not an isolated recording; it is one modality in a multi-modal health monitoring workflow.

Voice capture may occur during a prompted phrase, spontaneous speech, a short speech sample, a breathing-related sound segment, a cough-related segment, or another audio interval associated with the monitoring session. The system may coordinate the audio interval with the image capture interval so that vocal features can be fused with facial, respiratory, and environmental features from the same session.

Voice preprocessing may include noise screening, clipping detection, silence detection, microphone availability detection, and signal-to-noise evaluation. If the voice signal is too noisy, too short, clipped, silent, or contaminated by interfering speakers, the system may mark the acoustic path as inadequate, reduce influence of the acoustic features, or prompt the user to provide a new sample.

Acoustic features may include pitch-related information, energy-related information, spectral features, timing features, pause information, speech-rate information, breathiness-related markers, cough-related markers, vocal stability, or other voice characteristics available from recorded audio. These features may indicate stress, fatigue, respiratory strain, neurological concern, emotional state, or other health-related conditions at a preliminary screening level.

The acoustic feature vector may be fused with the respiratory feature vector. When both voice and video suggest respiratory concern, the DrAI module may increase a corresponding preliminary risk weight. When voice data are unreliable but respiratory video is reliable, the voice modality may be masked. When both voice and video are unreliable, the system may request repeat capture or generate an insufficient-data output.

Voice data can be privacy-sensitive. The system may process voice locally, discard raw audio after feature extraction, transmit only feature-derived information, or require authorization before transmitting any voice-related data. These options support practical deployment of the screen-based monitoring workflow without changing the core invention.

Voice embodiment 1 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 2 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 3 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 4 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 5 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 6 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 7 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 8 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 9 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

Voice embodiment 10 illustrates that the system may extract different acoustic feature subsets depending on microphone quality, capture duration, and prompt type. A short phrase may emphasize speech rate, pauses, and energy; a longer phrase may support spectral and timing features; and an inadequate phrase may be marked by the quality gate. In each case, the voice data are used as one input to DrAI fusion rather than as an independent definitive diagnosis.

### Environmental Particulate Interface Embodiments

The environmental subsystem detects or receives environmental particulate matter information through embedded miniature sensors or an environmental sensor interface associated with the device. The particulate information may indicate potential environmental health impacts in the immediate environment of the user or in the environment associated with the device at the time of monitoring.

Environmental feature processing may include receiving particulate readings, screening readings for validity, normalizing sensor values, associating the environmental information with the capture time of facial, respiratory, and voice data, and assigning an environmental quality flag. The environmental data need not provide a stand-alone medical conclusion; it serves as one source of contextual information for DrAI fusion.

The environmental interface may receive particulate information from an embedded sensor, a connected sensor, a smart-device sensor interface, or another device-associated source. The claims need not require a particular sensor manufacturer or measurement protocol. What is important for the present disclosure is that particulate matter information is received and used with facial, respiratory, and voice features during the monitoring session.

Environmental particulate information may support a preliminary warning when combined with respiratory or vocal features. For example, elevated particulate matter may provide context for cough markers, breathiness, or respiratory video irregularity. If environmental particulate information is unavailable or invalid, the quality gate may mark the environmental feature as missing or low confidence rather than treating the missing data as normal.

The environmental subsystem may store a sensor adequacy flag or source validity indicator with the session output. The flag allows the DrAI module to distinguish a reliable particulate context from an unavailable or questionable reading. This improves robustness and supports a more meaningful preliminary non-diagnostic output.

Environmental embodiment 1 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

Environmental embodiment 2 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

Environmental embodiment 3 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

Environmental embodiment 4 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

Environmental embodiment 5 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

Environmental embodiment 6 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

Environmental embodiment 7 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

Environmental embodiment 8 may associate particulate readings with the same monitoring interval used for facial, respiratory, and voice capture. The system may normalize the environmental feature, determine whether the reading is current enough for the session, and decide whether the feature should be fused, down-weighted, or excluded. This keeps the environmental component tied to the original particulate-analysis disclosure rather than expanding into unrelated environmental services.

### Signal-Quality Gate and Failure-State Logic

A signal-quality gate determines whether visual, acoustic, and environmental inputs are adequate for analysis. The quality gate may evaluate face visibility, lighting, image stability, frame adequacy, respiratory-region visibility, microphone signal quality, noise level, speech presence, sensor availability, sensor validity, and whether particulate readings are available for the monitoring session.

When the quality gate indicates inadequate data, the system may request a repeat capture, omit a feature, reduce confidence, mask a feature, or produce an output stating that the assessment is limited. The quality gate supports reliable operation and reduces the risk of over-stating a health assessment based on poor data. The output may therefore be a repeat-scan request rather than a health-risk conclusion.

Visual quality factors may include whether the face is visible, whether the relevant respiratory movement region is visible, whether lighting is adequate, whether image blur is present, whether camera movement is excessive, and whether the capture window contains sufficient frames. The same video stream may be suitable for facial analysis but not respiratory analysis, or vice versa, and the gate may assign separate flags for those conditions.

Acoustic quality factors may include speech presence, background noise, microphone clipping, audio duration, and signal-to-noise adequacy. Environmental quality factors may include sensor availability, sensor validity, and whether particulate readings are present for the monitoring session. Quality information may be stored with the assessment output so that the user or an authorized recipient can understand whether the result was based on adequate input.

A failure state may be generated when the system cannot produce a reliable preliminary assessment. The failure state may indicate insufficient video, insufficient voice, unavailable environmental data, conflicting features, or a need for repeat capture. This failure-state handling is a technical safeguard that supports real-world use and prevents low-quality input from being treated as high-quality health evidence.

Quality-gate embodiment 1 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 2 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 3 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 4 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 5 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 6 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 7 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 8 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 9 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 10 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

Quality-gate embodiment 11 may apply a particular combination of visual, acoustic, and environmental adequacy checks before DrAI fusion. If a signal fails but the remaining modalities are sufficient, the system may proceed with a reduced-confidence output. If a critical signal fails, the system may request repeat capture. If modality outputs conflict, the system may generate a limited preliminary state or a reason code rather than an unsupported definitive conclusion.

### Multi-Modal DrAI Fusion and Confidence Weighting

The integration subsystem receives facial feature vectors, respiratory feature vectors, vocal feature vectors, and environmental feature vectors. These vectors may be temporally associated within a monitoring session and provided to a multi-modal DrAI model. The system is not merely collecting independent data streams; it combines the feature sets to generate a real-time assessment and early warning output.

Temporal association may align facial video frames, respiratory video frames, voice samples, and environmental readings within a selected monitoring interval. The selected interval may be short enough to support real-time monitoring and long enough to extract respiratory and voice features. Alignment may be based on session start time, capture window, prompt time, frame time, audio time, or environmental reading time.

The DrAI module may apply confidence weighting to the extracted features. Features derived from high-quality data may receive higher influence, while features derived from inadequate data may be down-weighted, masked, or excluded. Confidence weighting supports a practical application of the multi-modal monitoring system because it prevents a single poor-quality input from dominating the preliminary assessment.

The DrAI fusion engine may be a deep learning model, a machine learning model, a statistical model, a rule-based model, or a hybrid model configured to combine the features and output a preliminary assessment or early warning. The model may use a combined representation of user state and environmental context rather than simply displaying separate raw data streams.

The fusion engine may generate an assessment score, a risk category, a repeat-capture state, an early warning alert, a quality-limited state, or an indication that no apparent risk was detected from available data. Any such output remains preliminary and non-diagnostic. The fused output may include reason information, confidence information, or quality flags tied to the session.

DrAI fusion embodiment 1 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 2 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 3 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 4 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 5 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 6 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 7 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 8 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 9 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 10 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 11 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 12 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

DrAI fusion embodiment 13 may combine facial, respiratory, vocal, and particulate features using a confidence-weighted representation. A respiratory feature may be strengthened when an acoustic respiratory indicator is also present. A facial stress or fatigue feature may be considered with voice timing and environmental context. A particulate feature may provide context for a respiratory or vocal indicator. These combinations illustrate the integrated monitoring loop without changing the original data sources.

### Preliminary Non-Diagnostic Assessment and Alert Output

A preliminary health assessment refers to a non-contact, screen-based assessment derived from available facial, respiratory, vocal, and environmental data. The assessment may indicate a current risk state, a need for repeat capture, or an early warning alert. The assessment is produced by the DrAI analysis module from data available during the session and is intended to support health risk management, user awareness, and timely follow-up when indicated.

An early warning alert may be generated when the DrAI module identifies a risk pattern based on the fused input data. The alert may be displayed on the device screen, delivered to the user device, or provided through a suitable notification interface associated with the smart device. The alert may include information indicating that the assessment is preliminary and non-diagnostic.

The original disclosure identifies real-time health assessments and alerts. In an embodiment, the processor generates the assessment during or shortly after the capture session and renders an output on the screen of the smart device or user device. Real-time delivery may include local display, device notification, audible notice, or network delivery to a user-authorized recipient.

In some embodiments, the real-time alert may be provided to an authorized user, caregiver, or clinician endpoint when such delivery is configured by the user or device. The endpoint notification is an extension of the original alert-delivery function and is limited to health assessment or warning communication. The system does not require a dedicated control center, rescue center, resource center, or unrelated institutional queue.

The system provides preliminary non-contact monitoring and early warning. The system does not replace physician examination, hospital evaluation, emergency medical services, continuous clinical monitoring, laboratory testing, medical imaging, pulse oximetry, electrocardiography, blood-pressure measurement, or other clinical diagnostic procedures. A no-alert or no-apparent-risk output does not establish absence of disease, absence of deterioration, or absence of emergency risk.

Output embodiment 1 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 2 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 3 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 4 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 5 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 6 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 7 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 8 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

Output embodiment 9 may display a real-time monitoring result, a repeat-capture request, a limited-data indication, a preliminary risk status, or an early warning alert. The user interface may avoid categorical diagnosis language and may present the result as preliminary. This supports safe practical use while preserving the technical purpose of converting screen-associated inputs into a DrAI assessment and alert.

### Local Processing, Software, and Implementation Details

The system may perform local feature extraction on the smart device to reduce exposure of raw data. In one embodiment, raw video or raw audio remains local and feature-derived information is used to generate the health assessment or alert. Privacy-related implementation may include permission prompts, local storage choices, secure transmission, or user consent before an alert is transmitted.

A monitoring session may generate a session output including a capture time, device identity, quality flags, extracted features, preliminary assessment, and alert state. Such output supports review of the session and is directly tied to the original monitoring and alert function. The session output is not required to include unrelated longitudinal databases, financial ledgers, rollback systems, or resource coordination systems.

The smart device may be a smartphone, tablet, laptop, desktop device, smart display, or other device screen equipped with or operatively associated with a camera, microphone, processor, memory, and environmental sensing interface. The device may execute instructions stored in memory to capture the relevant data, apply the signal-quality gate, extract the multi-modal features, run the DrAI module, and output the real-time assessment or alert.

The monitoring functions may be implemented as software instructions executed by one or more processors of the smart device or by a remote processor communicatively coupled to the device. A non-transitory computer-readable medium may store the instructions. The instructions may be distributed as part of an application, embedded device software, or another processor-readable program associated with the screen-based device.

A DrAI model may be trained or configured to recognize relationships among facial, respiratory, vocal, and environmental features. During use, the model receives feature vectors from the active monitoring session and generates a preliminary assessment or early warning status. The output may be constrained by non-diagnostic safety rules and by quality flags derived from the signal-quality gate.

Implementation embodiment 1 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 2 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 3 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 4 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 5 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 6 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 7 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 8 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 9 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 10 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

Implementation embodiment 11 may perform all analysis locally, may perform selected analysis remotely, or may use a hybrid arrangement in which feature extraction is local and DrAI fusion is remote. In each case, the system remains tied to the screen-associated camera, microphone, environmental interface, quality gate, multi-modal feature extraction, and preliminary output. The implementation choice does not convert the invention into an unrelated communication or record-management system.

### Advantages and Technical Distinctions

The disclosed system can improve convenience by using a smartphone or device screen that the user already possesses. It avoids requiring additional user-worn hardware and enables non-contact capture of multiple data types during a screen-based session. This can make preliminary monitoring more available in ordinary daily contexts.

The system also provides a multi-modal assessment because facial, respiratory, vocal, and environmental particulate inputs are considered together. This is more useful than a single-modal application that separately analyzes only video, only voice, or only environmental data. The quality gate further improves robustness by allowing unreliable features to be reduced, masked, or excluded.

Contact sensors may be inconvenient, forgotten, incorrectly positioned, or unavailable. In contrast, the disclosed system can operate through a screen-associated camera, microphone, and environmental sensor interface without requiring the user to attach a sensor to the body. The non-contact design is particularly useful for frequent checks, wellness monitoring, and early warning use cases.

An ordinary video call transmits images and sound for human viewing, but it does not necessarily extract facial, respiratory, voice, and environmental features and fuse them through a DrAI module to produce a preliminary assessment or early warning. The disclosed system provides structured feature extraction and analysis, thereby converting a screen-based interaction into a technical health monitoring workflow.

A single-source monitor may analyze only environmental particulate matter, only voice, or only video. The disclosed system integrates these sources so that environmental context, facial features, respiratory movement, and voice features can be considered together. This multi-modal structure may improve robustness because an alert can be based on more than one type of input and quality flags can reduce the influence of unreliable signals.

### Detailed Operation of the Screen-Based Monitoring Loop

Detailed operation step 1. A device screen displays a monitoring prompt to the user. The prompt may instruct the user to look toward the screen and remain within a capture window. The device prepares the camera, microphone, environmental sensor interface, processor, and memory for a synchronized monitoring session. This step is part of the same screen-based non-contact monitoring workflow and cooperates with the other steps to support a concrete system, method, and computer-readable-medium implementation.

Detailed operation step 2. The screen-associated camera captures video frames. The frames may include facial images and visible body-region movement from which respiratory movement can be derived. The microphone records voice data during the same session or a related session interval, and the environmental interface receives particulate matter data. This step is part of the same screen-based non-contact monitoring workflow and cooperates with the other steps to support a concrete system, method, and computer-readable-medium implementation.

Detailed operation step 3. The system evaluates whether the captured inputs are adequate. The visual component is checked for face visibility, sufficient lighting, frame adequacy, respiratory-region visibility, and movement stability. The audio component is checked for signal clarity, and the environmental component is checked for sensor validity. This step is part of the same screen-based non-contact monitoring workflow and cooperates with the other steps to support a concrete system, method, and computer-readable-medium implementation.

Detailed operation step 4. Facial features are extracted from the video frames. The features may include complexion, expressions, micro-expressions, and eye movements. Respiratory features are extracted by applying EVM or video motion processing to visible respiratory movement. This step is part of the same screen-based non-contact monitoring workflow and cooperates with the other steps to support a concrete system, method, and computer-readable-medium implementation.

Detailed operation step 5. Voice features are extracted from the recorded audio. Environmental features are derived from particulate matter readings. Each feature set may include a confidence value or quality flag so that the DrAI module can treat the feature in accordance with its reliability. This step is part of the same screen-based non-contact monitoring workflow and cooperates with the other steps to support a concrete system, method, and computer-readable-medium implementation.

Detailed operation step 6. The DrAI module fuses the extracted facial, respiratory, voice, and environmental feature sets. The fusion may include temporal alignment, normalization, confidence weighting, feature masking, and model-based or rules-based analysis. This step is part of the same screen-based non-contact monitoring workflow and cooperates with the other steps to support a concrete system, method, and computer-readable-medium implementation.

Detailed operation step 7. The fused representation is used to generate a preliminary health assessment, an early warning status, or a repeat-capture state. The output is provided through the screen or another authorized device output and remains preliminary and non-diagnostic. This step is part of the same screen-based non-contact monitoring workflow and cooperates with the other steps to support a concrete system, method, and computer-readable-medium implementation.

Detailed operation step 8. If the assessment indicates no alert based on available data, the device may state that no apparent risk was detected while also indicating that the output is not a medical clearance. If a risk pattern is detected, the device may display an early warning and may notify an authorized recipient if configured by the user. This step is part of the same screen-based non-contact monitoring workflow and cooperates with the other steps to support a concrete system, method, and computer-readable-medium implementation.

### Claim Support Mapping

The system claims are supported by the screen-based camera, integrated microphone, environmental sensors or environmental sensing interface, processor, memory, and DrAI module originally disclosed. The claims organize those elements into a concrete system having data capture, feature extraction, signal-quality gating, multi-modal fusion, and preliminary output functions. The system structure is intentionally focused on the original screen-based non-contact health monitoring disclosure.

The method claims are supported by the original disclosure of capturing facial videos, respiratory movement, speech frequencies or voice data, and particulate matter and analyzing them using DrAI. The method recites concrete steps for screen prompting, capture, EVM or video motion processing, voice feature extraction, environmental sensing, quality gating, fusion, and alert output. The method avoids unsupported clinical diagnosis or treatment steps.

The computer-readable-medium claims are supported because the system is implemented by smart devices and DrAI analysis executed by processors. Instructions may cause the processor to control the screen-associated camera, microphone, environmental sensor interface, feature extraction, signal-quality gate, fusion, and alert display. This claim category protects application and software implementations while remaining tied to the originally disclosed screen-based data acquisition and DrAI analysis functions.

The dependent claims provide fallback positions grounded in the original disclosure. Facial dependent claims recite complexion, expression, micro-expression, and eye movement. Respiratory dependent claims recite rate, depth, rhythm, confidence, region of interest, temporal filtering, EVM, and feature-vector generation. Voice dependent claims recite pitch, energy, spectral, timing, pause, and speech-rate features. Environmental dependent claims recite particulate matter information. Quality dependent claims recite adequacy checks, repeat capture, masking, exclusion, and non-diagnostic indications.

The claims avoid multiple dependent format and maintain a clear hierarchy. Claim 1 is a system claim directed to the smart device and processing modules. Claim 12 is a method claim directed to the monitoring workflow. Claim 20 is a non-transitory computer-readable medium claim directed to processor-executable instructions. Claims 2-11 depend from the system claim, and claims 13-19 depend from the method claim.

### BRIEF DESCRIPTION OF THE DRAWINGS

FIG. 1 is described in detail below. FIG. 1 illustrates an overall screen-based DrAI health monitoring node including a screen device, image sensor, microphone, environmental interface, feature extraction modules, signal-quality gate, DrAI fusion engine, and preliminary non-diagnostic health-risk output. The figure shows that the invention is a coordinated technical loop rather than an isolated camera, audio recorder, or particulate monitor.

FIG. 2 is described in detail below. FIG. 2 illustrates a session activation and non-contact acquisition workflow. A user before a screen receives a session activation prompt, enters a non-contact capture window, and provides synchronized image, voice, and environmental data. A quality adequacy check determines whether the system proceeds to analysis or requests repeat capture.

FIG. 3 is described in detail below. FIG. 3 illustrates facial feature extraction using screen-associated imaging. Captured face frames are processed to identify face, cheek, eye, periocular, lip, and expression regions. The system derives complexion, color, expression, fatigue-related, and eye-movement features and organizes them into a facial feature vector.

FIG. 4 is described in detail below. FIG. 4 illustrates respiratory micro-motion extraction from video. Video frames are processed to select a chest, neck, face, or other visible region of interest. Temporal filtering and Eulerian Video Magnification or optical flow are used to extract a motion waveform and produce respiratory rate, rhythm, depth, or confidence indicators.

FIG. 5 is described in detail below. FIG. 5 illustrates vocal acoustic feature extraction. A speech, cough, or breath segment is received by the microphone, screened for noise, and processed to derive pitch, jitter, shimmer, speech-rate, pause, cough, wheeze, or related acoustic features. These features are organized into a vocal feature vector for DrAI fusion.

FIG. 6 is described in detail below. FIG. 6 illustrates environmental particulate signal acquisition. Particulate matter and optional related environmental information are received by an environmental signal interface. Valid environmental readings are converted into an environmental feature vector and provided as context input to the DrAI analysis module.

FIG. 7 is described in detail below. FIG. 7 illustrates a signal-quality gate and repeat-scan logic. Visual adequacy, acoustic adequacy, and sensor adequacy are evaluated. Adequate data proceed to fusion, low-confidence data may be downgraded or masked, and failed data may result in a repeat-scan request or limited output.

FIG. 8 is described in detail below. FIG. 8 illustrates multi-modal DrAI fusion and confidence weighting. Facial, respiratory, vocal, and environmental features are supplied to a confidence-weighted DrAI fusion engine. The engine may perform feature downgrade, masking, cross-modal consistency evaluation, and generation of a fused risk state.

FIG. 9 is described in detail below. FIG. 9 illustrates preliminary health-risk output categories. A fused risk state may produce no apparent risk, repeat scan, authorized notification, early warning alert, non-diagnostic notice, or device screen output. The figure emphasizes that the output remains preliminary and non-diagnostic.

FIG. 10 is described in detail below. FIG. 10 illustrates a privacy boundary and feature-derived output. Raw video, audio, and sensor inputs may be processed locally into feature-derived indicators and preliminary assessment records. Authorized output is controlled so that the system can reduce exposure of raw data while producing an assessment or alert.

FIG. 11 is described in detail below. FIG. 11 illustrates user device and authorized recipient output. A preliminary assessment or alert may be displayed on the user device screen, directed to an authorized caregiver or clinician endpoint, or converted into a repeat-capture prompt. The output remains within the non-diagnostic boundary.

FIG. 12 is described in detail below. FIG. 12 illustrates a conservative finite screen-based monitoring workflow. The screen prompt initiates capture of video, voice, and particulate data. The quality gate determines whether feature extraction and DrAI fusion should proceed, and the system produces a preliminary assessment, alert, or repeat-capture output.

FIG. 13 is described in detail below. FIG. 13 illustrates a monitoring session data structure. A session may include session time, device information, quality flags, facial features, respiratory features, vocal features, environmental features, preliminary assessment, and a screen-based monitoring record. The record supports the monitoring workflow without requiring unrelated databases.

FIG. 14 is described in detail below. FIG. 14 illustrates calibration, threshold, and validation management. Camera calibration, microphone calibration, sensor calibration, versioned thresholds, validation rules, forbidden output rules, and auditable model state may support safe and reliable use of the DrAI module within the screen-based monitoring system.

FIG. 15 is described in detail below. FIG. 15 illustrates assistive use and the non-diagnostic boundary. A preliminary output is not a final diagnosis, not continuous clinical monitoring, and not medical clearance. Users may be advised to seek care when symptoms are concerning, while the system provides assistive early warning only.

FIG. 16 is described in detail below. FIG. 16 illustrates a screen-based multi-modal monitoring implementation structure. The figure shows the same monitoring core as a system, method, and computer-readable-medium implementation using facial, respiratory, vocal, and environmental features, signal-quality operations, DrAI fusion, alert output, and a no-new-matter conservative boundary.

### DETAILED DESCRIPTION OF THE DRAWINGS

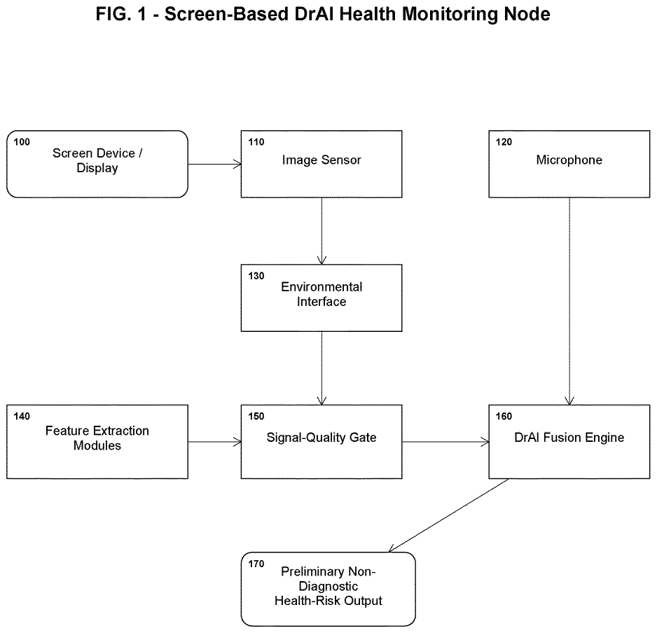

Referring to FIG. 1, FIG. 1 illustrates an overall screen-based DrAI health monitoring node including a screen device, image sensor, microphone, environmental interface, feature extraction modules, signal-quality gate, DrAI fusion engine, and preliminary non-diagnostic health-risk output. The figure shows that the invention is a coordinated technical loop rather than an isolated camera, audio recorder, or particulate monitor. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

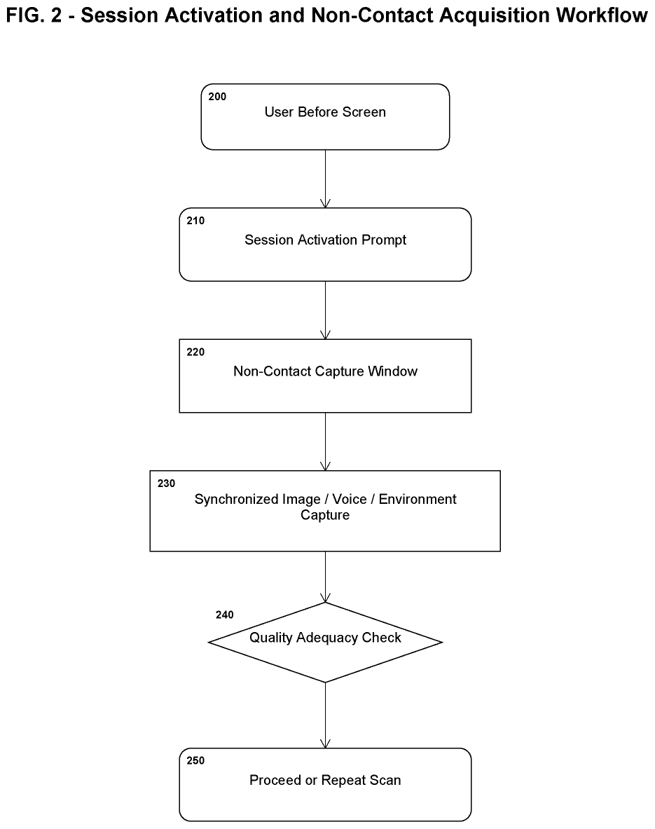

Referring to FIG. 2, FIG. 2 illustrates a session activation and non-contact acquisition workflow. A user before a screen receives a session activation prompt, enters a non-contact capture window, and provides synchronized image, voice, and environmental data. A quality adequacy check determines whether the system proceeds to analysis or requests repeat capture. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

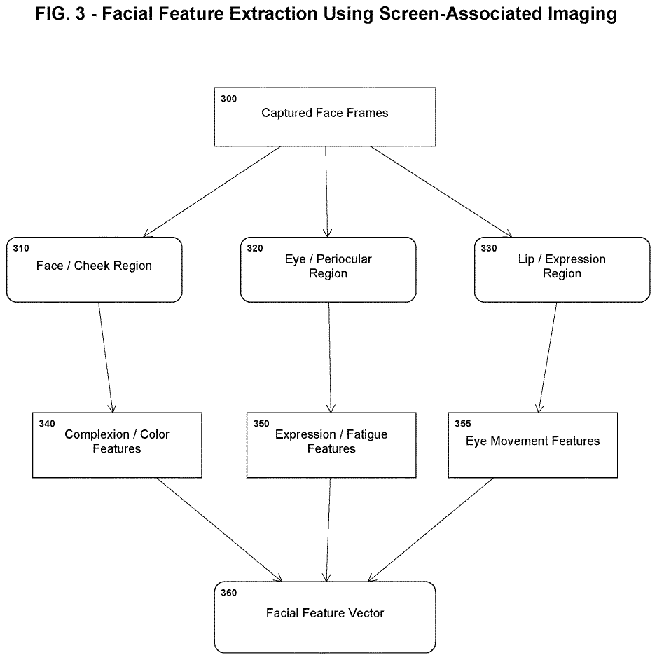

Referring to FIG. 3, FIG. 3 illustrates facial feature extraction using screen-associated imaging. Captured face frames are processed to identify face, cheek, eye, periocular, lip, and expression regions. The system derives complexion, color, expression, fatigue-related, and eye-movement features and organizes them into a facial feature vector. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

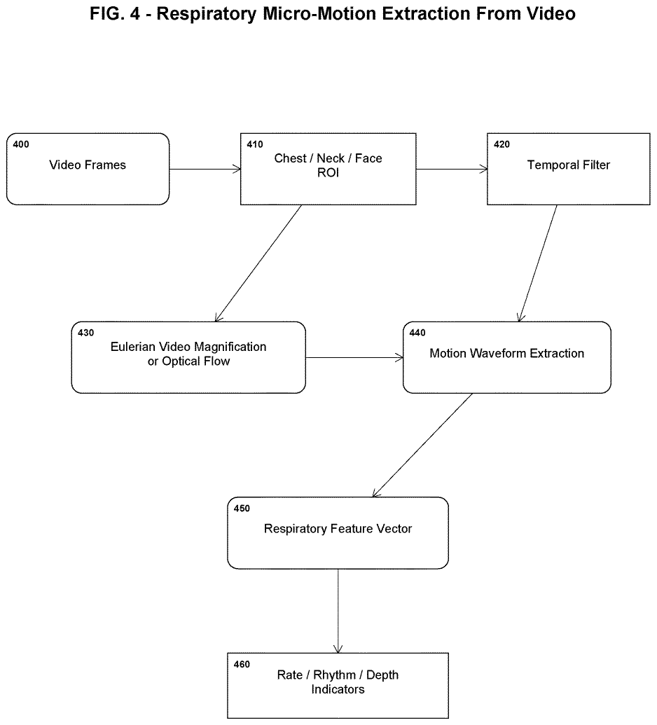

Referring to FIG. 4, FIG. 4 illustrates respiratory micro-motion extraction from video. Video frames are processed to select a chest, neck, face, or other visible region of interest. Temporal filtering and Eulerian Video Magnification or optical flow are used to extract a motion waveform and produce respiratory rate, rhythm, depth, or confidence indicators. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

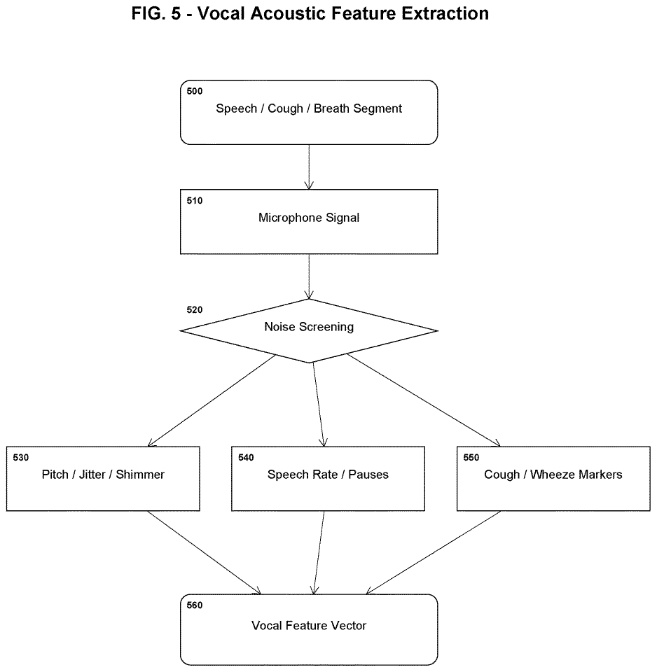

Referring to FIG. 5, FIG. 5 illustrates vocal acoustic feature extraction. A speech, cough, or breath segment is received by the microphone, screened for noise, and processed to derive pitch, jitter, shimmer, speech-rate, pause, cough, wheeze, or related acoustic features. These features are organized into a vocal feature vector for DrAI fusion. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

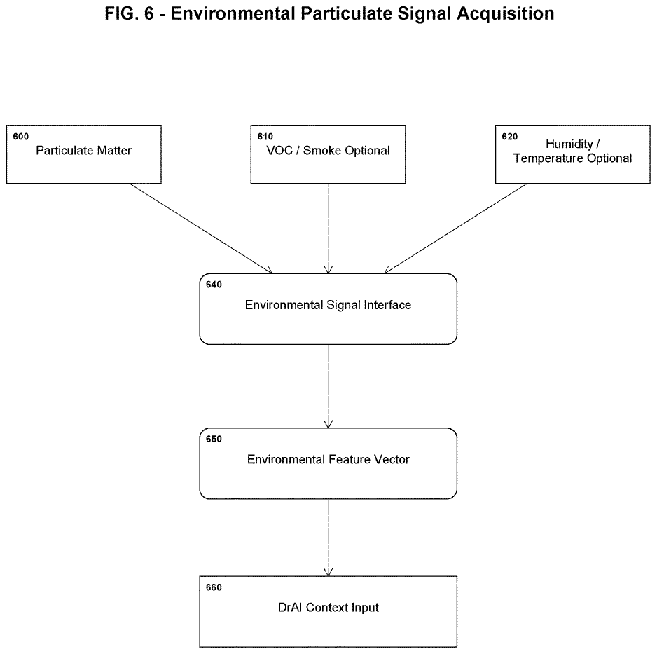

Referring to FIG. 6, FIG. 6 illustrates environmental particulate signal acquisition. Particulate matter and optional related environmental information are received by an environmental signal interface. Valid environmental readings are converted into an environmental feature vector and provided as context input to the DrAI analysis module. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

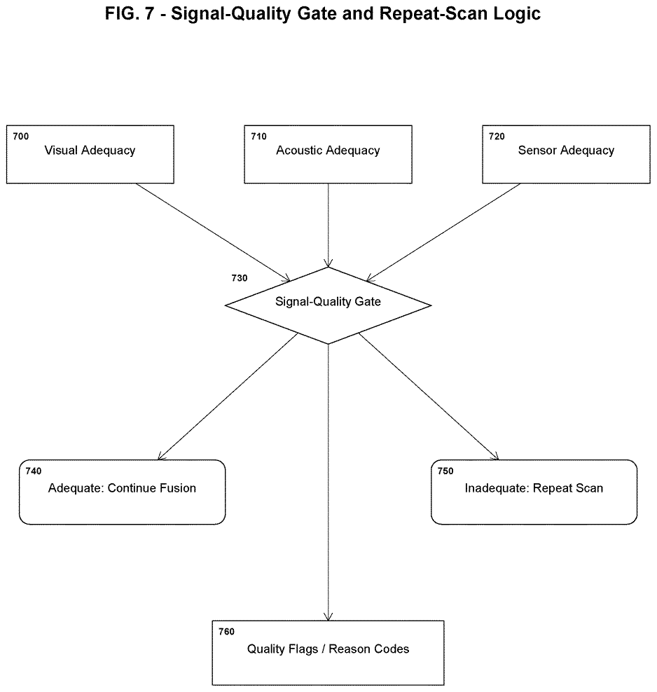

Referring to FIG. 7, FIG. 7 illustrates a signal-quality gate and repeat-scan logic. Visual adequacy, acoustic adequacy, and sensor adequacy are evaluated. Adequate data proceed to fusion, low-confidence data may be downgraded or masked, and failed data may result in a repeat-scan request or limited output. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

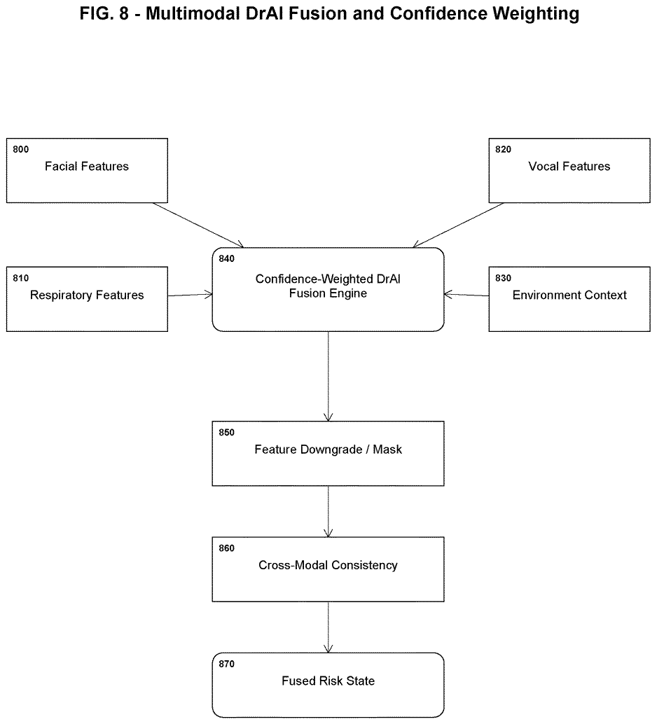

Referring to FIG. 8, FIG. 8 illustrates multi-modal DrAI fusion and confidence weighting. Facial, respiratory, vocal, and environmental features are supplied to a confidence-weighted DrAI fusion engine. The engine may perform feature downgrade, masking, cross-modal consistency evaluation, and generation of a fused risk state. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

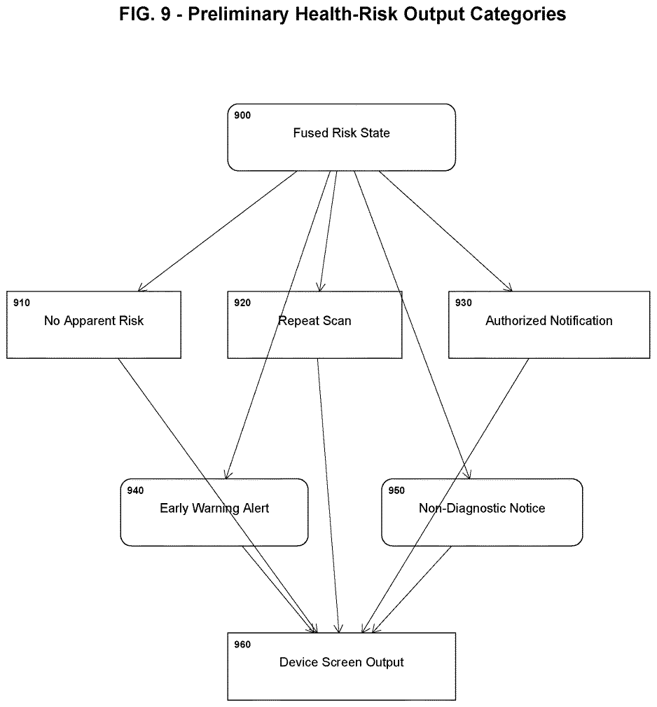

Referring to FIG. 9, FIG. 9 illustrates preliminary health-risk output categories. A fused risk state may produce no apparent risk, repeat scan, authorized notification, early warning alert, non-diagnostic notice, or device screen output. The figure emphasizes that the output remains preliminary and non-diagnostic. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

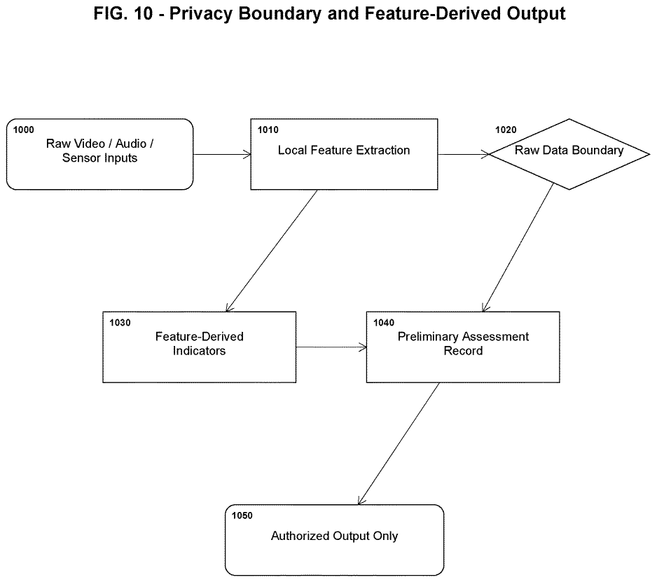

Referring to FIG. 10, FIG. 10 illustrates a privacy boundary and feature-derived output. Raw video, audio, and sensor inputs may be processed locally into feature-derived indicators and preliminary assessment records. Authorized output is controlled so that the system can reduce exposure of raw data while producing an assessment or alert. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

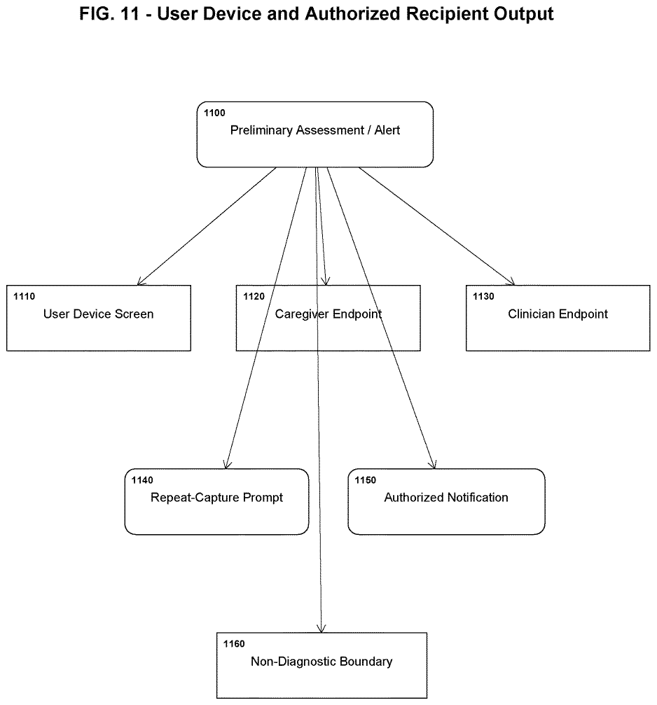

Referring to FIG. 11, FIG. 11 illustrates user device and authorized recipient output. A preliminary assessment or alert may be displayed on the user device screen, directed to an authorized caregiver or clinician endpoint, or converted into a repeat-capture prompt. The output remains within the non-diagnostic boundary. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

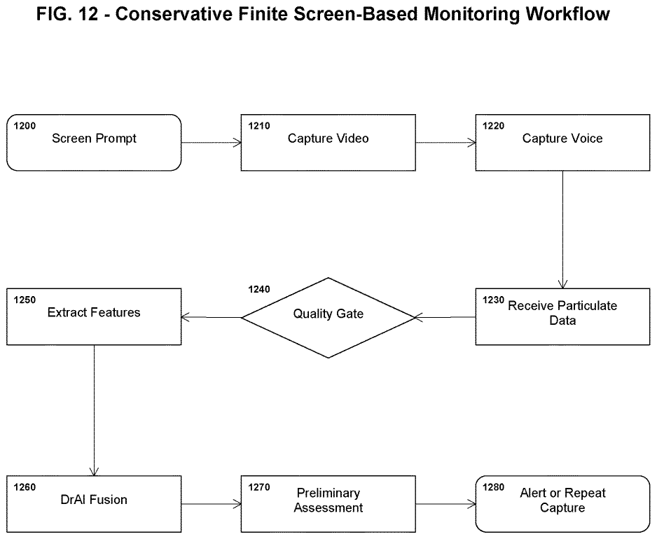

Referring to FIG. 12, FIG. 12 illustrates a conservative finite screen-based monitoring workflow. The screen prompt initiates capture of video, voice, and particulate data. The quality gate determines whether feature extraction and DrAI fusion should proceed, and the system produces a preliminary assessment, alert, or repeat-capture output. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

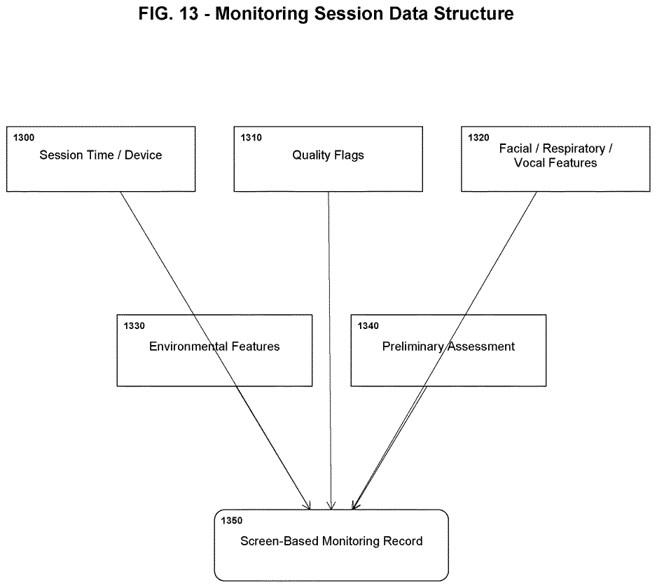

Referring to FIG. 13, FIG. 13 illustrates a monitoring session data structure. A session may include session time, device information, quality flags, facial features, respiratory features, vocal features, environmental features, preliminary assessment, and a screen-based monitoring record. The record supports the monitoring workflow without requiring unrelated databases. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

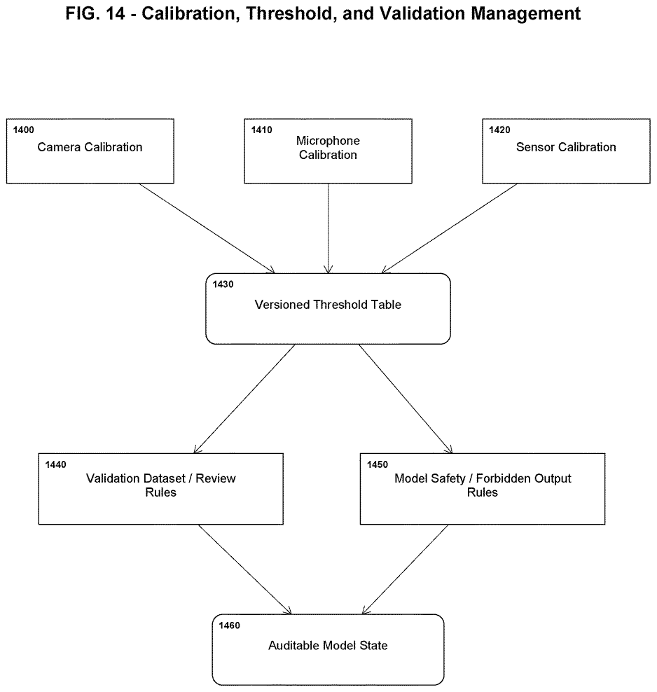

Referring to FIG. 14, FIG. 14 illustrates calibration, threshold, and validation management. Camera calibration, microphone calibration, sensor calibration, versioned thresholds, validation rules, forbidden output rules, and auditable model state may support safe and reliable use of the DrAI module within the screen-based monitoring system. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

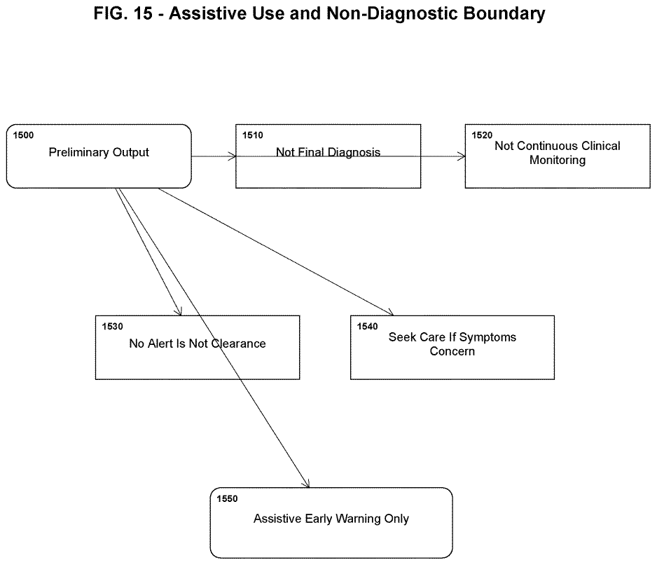

Referring to FIG. 15, FIG. 15 illustrates assistive use and the non-diagnostic boundary. A preliminary output is not a final diagnosis, not continuous clinical monitoring, and not medical clearance. Users may be advised to seek care when symptoms are concerning, while the system provides assistive early warning only. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

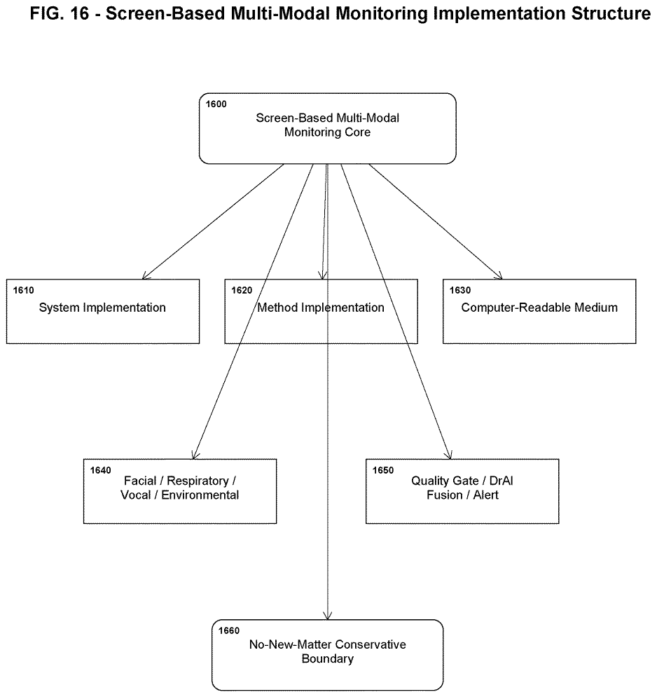

Referring to FIG. 16, FIG. 16 illustrates a screen-based multi-modal monitoring implementation structure. The figure shows the same monitoring core as a system, method, and computer-readable-medium implementation using facial, respiratory, vocal, and environmental features, signal-quality operations, DrAI fusion, alert output, and a no-new-matter conservative boundary. The drawing is schematic and is intended to support understanding of the disclosed screen-based non-contact monitoring workflow. The figure may omit ordinary device details not necessary for understanding the facial, respiratory, vocal, environmental, quality-gate, DrAI fusion, preliminary assessment, and early-warning functions.

### REPRESENTATIVE EXAMPLES

Example 1. In a smartphone captures facial video, respiratory movement, voice data, and particulate information and outputs a repeat-capture request because lighting is inadequate. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 2. In a tablet captures adequate facial and voice data but weak respiratory motion and therefore reduces respiratory confidence before DrAI fusion. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 3. In a smart display receives elevated particulate matter information and fuses the environmental context with voice and respiratory features during a screen-based session. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 4. In a laptop application processes voice acoustic features and facial expression features while the user faces the camera and provides a preliminary non-diagnostic output. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 5. In a device performs EVM on a visible respiratory region and uses the resulting waveform with acoustic pause features and complexion features to generate an early warning. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 6. In a local-processing embodiment extracts feature-derived indicators from video and audio while keeping raw input on the device. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 7. In a repeat-capture embodiment prompts the user to improve positioning, lighting, or speech sample before another monitoring attempt. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 8. In a non-diagnostic output embodiment displays a preliminary assessment and a notice that the output is not medical clearance or a definitive diagnosis. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 9. In an authorized notification embodiment delivers a preliminary alert to a user-authorized caregiver or clinician endpoint without requiring a control center. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

Example 10. In a software embodiment stores instructions that control camera capture, microphone recording, environmental input, signal-quality gating, feature extraction, DrAI fusion, and screen alert display. This example uses only the screen-associated camera, microphone, environmental particulate interface, processor, signal-quality gate, DrAI fusion engine, and preliminary output functions disclosed for the invention. The example is illustrative and does not limit the claims to a particular device brand, software title, or commercial deployment name.

### Additional Facial Implementation Details

Facial implementation detail 1. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 1A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

Facial implementation detail 2. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 2A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

Facial implementation detail 3. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 3A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

Facial implementation detail 4. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 4A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

Facial implementation detail 5. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 5A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

Facial implementation detail 6. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 6A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

Facial implementation detail 7. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 7A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

Facial implementation detail 8. The processor may divide the captured face into stable regions for complexion, expression, micro-expression, and eye-movement analysis. Region stability may be evaluated over the same monitoring interval used for respiratory and voice capture. If a region is not stable enough, the corresponding feature may be assigned a lower confidence value before DrAI fusion. This supports the disclosed facial analysis without adding a separate diagnostic method.

Facial implementation detail 8A. The system may compare successive frames to identify temporal changes in a face region while also considering illumination and camera quality. Because the disclosed assessment is preliminary and non-diagnostic, the extracted facial feature is used as one element in a multi-modal representation rather than as an independent conclusion. The quality gate may control whether the facial feature is accepted, down-weighted, or repeated.

### Additional Respiratory Video Implementation Details

Respiratory processing detail 1. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 1A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

Respiratory processing detail 2. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 2A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

Respiratory processing detail 3. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 3A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

Respiratory processing detail 4. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 4A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

Respiratory processing detail 5. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 5A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

Respiratory processing detail 6. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 6A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

Respiratory processing detail 7. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 7A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

Respiratory processing detail 8. The video respiratory path may begin with a visible-region test, proceed to selection of one or more candidate respiratory regions, apply temporal filtering, and then apply EVM or another video motion processing operation. The resulting waveform may be examined for periodicity, artifact, and confidence before a respiratory feature vector is released to the DrAI module.

Respiratory processing detail 8A. The system may use a fallback region when a preferred region is not visible. For example, when a chest region is not available in the frame, the processor may use neck, shoulder, lower-face, or other visible movement information if adequate. This fallback remains within the original non-contact respiratory video analysis disclosure because it relies on screen-captured respiratory movement rather than a body-contact sensor.

### Additional Voice and Microphone Implementation Details

Acoustic implementation detail 1. The microphone path may create a voice feature vector only after screening for speech presence, noise, clipping, and adequate duration. The feature vector may include timing, pause, pitch, energy, spectral, or speech-rate characteristics. These features may be associated with emotional state, psychological stress, or respiratory and neurological indicators at a preliminary level.

Acoustic implementation detail 1A. The system may prompt a user to repeat a short phrase when the audio signal is inadequate. If the audio remains inadequate, the DrAI module may mask the acoustic path and proceed with a lower-confidence multi-modal assessment when the visual and environmental paths are sufficient. This supports reliable use without requiring a special external microphone or a definitive voice diagnosis.

Acoustic implementation detail 2. The microphone path may create a voice feature vector only after screening for speech presence, noise, clipping, and adequate duration. The feature vector may include timing, pause, pitch, energy, spectral, or speech-rate characteristics. These features may be associated with emotional state, psychological stress, or respiratory and neurological indicators at a preliminary level.

Acoustic implementation detail 2A. The system may prompt a user to repeat a short phrase when the audio signal is inadequate. If the audio remains inadequate, the DrAI module may mask the acoustic path and proceed with a lower-confidence multi-modal assessment when the visual and environmental paths are sufficient. This supports reliable use without requiring a special external microphone or a definitive voice diagnosis.

Acoustic implementation detail 3. The microphone path may create a voice feature vector only after screening for speech presence, noise, clipping, and adequate duration. The feature vector may include timing, pause, pitch, energy, spectral, or speech-rate characteristics. These features may be associated with emotional state, psychological stress, or respiratory and neurological indicators at a preliminary level.

Acoustic implementation detail 3A. The system may prompt a user to repeat a short phrase when the audio signal is inadequate. If the audio remains inadequate, the DrAI module may mask the acoustic path and proceed with a lower-confidence multi-modal assessment when the visual and environmental paths are sufficient. This supports reliable use without requiring a special external microphone or a definitive voice diagnosis.

Acoustic implementation detail 4. The microphone path may create a voice feature vector only after screening for speech presence, noise, clipping, and adequate duration. The feature vector may include timing, pause, pitch, energy, spectral, or speech-rate characteristics. These features may be associated with emotional state, psychological stress, or respiratory and neurological indicators at a preliminary level.

Acoustic implementation detail 4A. The system may prompt a user to repeat a short phrase when the audio signal is inadequate. If the audio remains inadequate, the DrAI module may mask the acoustic path and proceed with a lower-confidence multi-modal assessment when the visual and environmental paths are sufficient. This supports reliable use without requiring a special external microphone or a definitive voice diagnosis.

Acoustic implementation detail 5. The microphone path may create a voice feature vector only after screening for speech presence, noise, clipping, and adequate duration. The feature vector may include timing, pause, pitch, energy, spectral, or speech-rate characteristics. These features may be associated with emotional state, psychological stress, or respiratory and neurological indicators at a preliminary level.

Acoustic implementation detail 5A. The system may prompt a user to repeat a short phrase when the audio signal is inadequate. If the audio remains inadequate, the DrAI module may mask the acoustic path and proceed with a lower-confidence multi-modal assessment when the visual and environmental paths are sufficient. This supports reliable use without requiring a special external microphone or a definitive voice diagnosis.

### Additional Environmental Particulate Implementation Details

Environmental implementation detail 1. The environmental sensing interface may receive particulate matter information associated with the capture interval. The processor may determine whether the reading is present, current, and valid enough for use. If the environmental signal is unavailable or unreliable, the environmental feature can be marked unavailable rather than treated as a normal reading.

Environmental implementation detail 1A. Particulate information is used as context for the DrAI assessment and does not by itself establish a medical condition. In the disclosed loop, the environmental feature is combined with facial, respiratory, and vocal features so that the preliminary alert may reflect both user-derived non-contact signals and the surrounding particulate context.

Environmental implementation detail 2. The environmental sensing interface may receive particulate matter information associated with the capture interval. The processor may determine whether the reading is present, current, and valid enough for use. If the environmental signal is unavailable or unreliable, the environmental feature can be marked unavailable rather than treated as a normal reading.

Environmental implementation detail 2A. Particulate information is used as context for the DrAI assessment and does not by itself establish a medical condition. In the disclosed loop, the environmental feature is combined with facial, respiratory, and vocal features so that the preliminary alert may reflect both user-derived non-contact signals and the surrounding particulate context.

Environmental implementation detail 3. The environmental sensing interface may receive particulate matter information associated with the capture interval. The processor may determine whether the reading is present, current, and valid enough for use. If the environmental signal is unavailable or unreliable, the environmental feature can be marked unavailable rather than treated as a normal reading.

Environmental implementation detail 3A. Particulate information is used as context for the DrAI assessment and does not by itself establish a medical condition. In the disclosed loop, the environmental feature is combined with facial, respiratory, and vocal features so that the preliminary alert may reflect both user-derived non-contact signals and the surrounding particulate context.

Environmental implementation detail 4. The environmental sensing interface may receive particulate matter information associated with the capture interval. The processor may determine whether the reading is present, current, and valid enough for use. If the environmental signal is unavailable or unreliable, the environmental feature can be marked unavailable rather than treated as a normal reading.

Environmental implementation detail 4A. Particulate information is used as context for the DrAI assessment and does not by itself establish a medical condition. In the disclosed loop, the environmental feature is combined with facial, respiratory, and vocal features so that the preliminary alert may reflect both user-derived non-contact signals and the surrounding particulate context.

### Additional Signal-Quality and Failure-State Details

Signal-quality implementation detail 1. The quality gate may generate separate visual, respiratory, acoustic, and environmental flags. A visual flag may apply to face visibility, an additional visual flag may apply to respiratory-region visibility, an acoustic flag may apply to microphone data, and an environmental flag may apply to particulate readings. Separate flags allow the system to preserve usable data while preventing unreliable data from dominating the output.

Signal-quality implementation detail 1A. The quality gate may produce a repeat-capture prompt, a limited-data indication, or a lower-confidence preliminary result. This behavior is particularly important because ordinary screen environments may include low light, hand movement, background speech, and unavailable particulate readings. The disclosed quality gate therefore provides a concrete technical safeguard for the DrAI Screen workflow.

Signal-quality implementation detail 2. The quality gate may generate separate visual, respiratory, acoustic, and environmental flags. A visual flag may apply to face visibility, an additional visual flag may apply to respiratory-region visibility, an acoustic flag may apply to microphone data, and an environmental flag may apply to particulate readings. Separate flags allow the system to preserve usable data while preventing unreliable data from dominating the output.

Signal-quality implementation detail 2A. The quality gate may produce a repeat-capture prompt, a limited-data indication, or a lower-confidence preliminary result. This behavior is particularly important because ordinary screen environments may include low light, hand movement, background speech, and unavailable particulate readings. The disclosed quality gate therefore provides a concrete technical safeguard for the DrAI Screen workflow.

Signal-quality implementation detail 3. The quality gate may generate separate visual, respiratory, acoustic, and environmental flags. A visual flag may apply to face visibility, an additional visual flag may apply to respiratory-region visibility, an acoustic flag may apply to microphone data, and an environmental flag may apply to particulate readings. Separate flags allow the system to preserve usable data while preventing unreliable data from dominating the output.

Signal-quality implementation detail 3A. The quality gate may produce a repeat-capture prompt, a limited-data indication, or a lower-confidence preliminary result. This behavior is particularly important because ordinary screen environments may include low light, hand movement, background speech, and unavailable particulate readings. The disclosed quality gate therefore provides a concrete technical safeguard for the DrAI Screen workflow.

Signal-quality implementation detail 4. The quality gate may generate separate visual, respiratory, acoustic, and environmental flags. A visual flag may apply to face visibility, an additional visual flag may apply to respiratory-region visibility, an acoustic flag may apply to microphone data, and an environmental flag may apply to particulate readings. Separate flags allow the system to preserve usable data while preventing unreliable data from dominating the output.

Signal-quality implementation detail 4A. The quality gate may produce a repeat-capture prompt, a limited-data indication, or a lower-confidence preliminary result. This behavior is particularly important because ordinary screen environments may include low light, hand movement, background speech, and unavailable particulate readings. The disclosed quality gate therefore provides a concrete technical safeguard for the DrAI Screen workflow.

Signal-quality implementation detail 5. The quality gate may generate separate visual, respiratory, acoustic, and environmental flags. A visual flag may apply to face visibility, an additional visual flag may apply to respiratory-region visibility, an acoustic flag may apply to microphone data, and an environmental flag may apply to particulate readings. Separate flags allow the system to preserve usable data while preventing unreliable data from dominating the output.

Signal-quality implementation detail 5A. The quality gate may produce a repeat-capture prompt, a limited-data indication, or a lower-confidence preliminary result. This behavior is particularly important because ordinary screen environments may include low light, hand movement, background speech, and unavailable particulate readings. The disclosed quality gate therefore provides a concrete technical safeguard for the DrAI Screen workflow.

### Additional DrAI Fusion and Output Details

Fusion implementation detail 1. Before DrAI fusion, the processor may normalize feature vectors and associate them with a monitoring time window. Confidence values may be attached to each modality. The DrAI module may then use the feature values and confidence values to generate a preliminary assessment, an early warning, a repeat-capture state, or a limited-data status.

Fusion implementation detail 1A. Multi-modal fusion can distinguish a single low-quality signal from a consistent pattern across modalities. For example, a respiratory video feature may be evaluated together with acoustic pause information and particulate context. The use of combined features provides a concrete technical difference from ordinary single-source monitoring and remains within the original DrAI multi-modal analysis disclosure.

Fusion implementation detail 2. Before DrAI fusion, the processor may normalize feature vectors and associate them with a monitoring time window. Confidence values may be attached to each modality. The DrAI module may then use the feature values and confidence values to generate a preliminary assessment, an early warning, a repeat-capture state, or a limited-data status.

Fusion implementation detail 2A. Multi-modal fusion can distinguish a single low-quality signal from a consistent pattern across modalities. For example, a respiratory video feature may be evaluated together with acoustic pause information and particulate context. The use of combined features provides a concrete technical difference from ordinary single-source monitoring and remains within the original DrAI multi-modal analysis disclosure.

Fusion implementation detail 3. Before DrAI fusion, the processor may normalize feature vectors and associate them with a monitoring time window. Confidence values may be attached to each modality. The DrAI module may then use the feature values and confidence values to generate a preliminary assessment, an early warning, a repeat-capture state, or a limited-data status.

Fusion implementation detail 3A. Multi-modal fusion can distinguish a single low-quality signal from a consistent pattern across modalities. For example, a respiratory video feature may be evaluated together with acoustic pause information and particulate context. The use of combined features provides a concrete technical difference from ordinary single-source monitoring and remains within the original DrAI multi-modal analysis disclosure.

Fusion implementation detail 4. Before DrAI fusion, the processor may normalize feature vectors and associate them with a monitoring time window. Confidence values may be attached to each modality. The DrAI module may then use the feature values and confidence values to generate a preliminary assessment, an early warning, a repeat-capture state, or a limited-data status.

Fusion implementation detail 4A. Multi-modal fusion can distinguish a single low-quality signal from a consistent pattern across modalities. For example, a respiratory video feature may be evaluated together with acoustic pause information and particulate context. The use of combined features provides a concrete technical difference from ordinary single-source monitoring and remains within the original DrAI multi-modal analysis disclosure.

Fusion implementation detail 5. Before DrAI fusion, the processor may normalize feature vectors and associate them with a monitoring time window. Confidence values may be attached to each modality. The DrAI module may then use the feature values and confidence values to generate a preliminary assessment, an early warning, a repeat-capture state, or a limited-data status.

Fusion implementation detail 5A. Multi-modal fusion can distinguish a single low-quality signal from a consistent pattern across modalities. For example, a respiratory video feature may be evaluated together with acoustic pause information and particulate context. The use of combined features provides a concrete technical difference from ordinary single-source monitoring and remains within the original DrAI multi-modal analysis disclosure.

### Additional Screen Interface and Alert Presentation Details

User-interface implementation detail 1. The screen may display capture instructions, progress status, quality messages, repeat-capture prompts, preliminary assessment outputs, early warning outputs, or non-diagnostic notices. The user interface supports the technical workflow by helping obtain adequate data and by communicating the preliminary nature of the output.

User-interface implementation detail 1A. A screen prompt may tell the user to improve lighting, move within the frame, speak again, or wait while the processor completes analysis. These prompts are acquisition and reliability functions. They do not add a treatment step and do not convert the preliminary assessment into a clinical diagnosis.

User-interface implementation detail 2. The screen may display capture instructions, progress status, quality messages, repeat-capture prompts, preliminary assessment outputs, early warning outputs, or non-diagnostic notices. The user interface supports the technical workflow by helping obtain adequate data and by communicating the preliminary nature of the output.

User-interface implementation detail 2A. A screen prompt may tell the user to improve lighting, move within the frame, speak again, or wait while the processor completes analysis. These prompts are acquisition and reliability functions. They do not add a treatment step and do not convert the preliminary assessment into a clinical diagnosis.

User-interface implementation detail 3. The screen may display capture instructions, progress status, quality messages, repeat-capture prompts, preliminary assessment outputs, early warning outputs, or non-diagnostic notices. The user interface supports the technical workflow by helping obtain adequate data and by communicating the preliminary nature of the output.

User-interface implementation detail 3A. A screen prompt may tell the user to improve lighting, move within the frame, speak again, or wait while the processor completes analysis. These prompts are acquisition and reliability functions. They do not add a treatment step and do not convert the preliminary assessment into a clinical diagnosis.

User-interface implementation detail 4. The screen may display capture instructions, progress status, quality messages, repeat-capture prompts, preliminary assessment outputs, early warning outputs, or non-diagnostic notices. The user interface supports the technical workflow by helping obtain adequate data and by communicating the preliminary nature of the output.

User-interface implementation detail 4A. A screen prompt may tell the user to improve lighting, move within the frame, speak again, or wait while the processor completes analysis. These prompts are acquisition and reliability functions. They do not add a treatment step and do not convert the preliminary assessment into a clinical diagnosis.

### CONCLUSION

The disclosed invention converts a smartphone or device screen environment into a non-contact, multi-modal DrAI health monitoring node. The system obtains facial, respiratory, vocal, and environmental particulate information, evaluates quality, extracts features, fuses the features, and outputs a preliminary non-diagnostic health assessment or real-time early warning alert. The combination provides a concrete technical implementation of the original DrAI Screen health monitoring disclosure.

The foregoing description is intended to explain the invention in enabling detail while preserving the no-new-matter boundary. Variations in device type, local or remote processing, model type, threshold configuration, signal-quality logic, and output interface may be used so long as the implementation remains within the claimed screen-based, non-contact, multi-modal health monitoring structure and operation.

1. A non-contact screen-based intelligent health monitoring system comprising: a smart device having a device screen, a screen-associated camera, an integrated microphone, one or more environmental sensing interfaces, one or more processors, and memory storing instructions; the screen-associated camera configured to capture facial image data and respiratory movement data of a user without physical contact with the user; the integrated microphone configured to record voice data of the user; the one or more environmental sensing interfaces configured to receive particulate matter information for an environment of the smart device; the one or more processors configured to apply a signal-quality gate to the facial image data, the respiratory movement data, the voice data, and the particulate matter information; extract facial features comprising complexion, expression, micro-expression, or eye-movement information; extract respiratory features from the respiratory movement data using Eulerian Video Magnification or video motion processing; extract acoustic features from the voice data; generate environmental features from the particulate matter information; and fuse the facial features, respiratory features, acoustic features, and environmental features using a DrAI analysis module to output a preliminary non-diagnostic health assessment or real-time early warning alert through the smart device.

2. The system of claim 1, wherein the screen-associated camera is a smartphone, tablet, computer, or smart-device camera positioned to capture the user while the user faces the device screen.

3. The system of claim 1, wherein the facial features include complexion information, facial expression information, micro-expression information, and eye-movement information used by the DrAI analysis module to evaluate stress, fatigue, or a potential health-risk indicator.

4. The system of claim 1, wherein the respiratory features include a respiratory rate, respiratory depth, respiratory rhythm, or respiratory movement confidence value derived from video frames without direct physical contact with the user.

5. The system of claim 1, wherein the acoustic features include pitch-related, energy-related, spectral, timing, pause, or speech-rate features extracted from the voice data.

6. The system of claim 1, wherein the environmental features include particulate matter concentration information associated with a capture interval of the facial image data, respiratory movement data, and voice data.

7. The system of claim 1, wherein the signal-quality gate is configured to determine visual adequacy, acoustic adequacy, and environmental sensor adequacy before the DrAI analysis module outputs the preliminary non-diagnostic health assessment or real-time early warning alert.

8. The system of claim 1, wherein the one or more processors are configured to reduce influence of, mask, or exclude a feature associated with an inadequate signal-quality flag.

9. The system of claim 1, wherein the preliminary non-diagnostic health assessment or real-time early warning alert is displayed on the device screen or delivered to an authorized user, caregiver, or clinician endpoint.

10. The system of claim 1, wherein the output includes a repeat-capture request when the signal-quality gate determines that captured image data, voice data, or environmental data are insufficient for reliable processing.

11. The system of claim 1, wherein the preliminary non-diagnostic health assessment or real-time early warning alert includes an indication that the output is not a definitive medical diagnosis.

12. A computer-implemented non-contact intelligent health monitoring method comprising: displaying, by a smart device screen, a monitoring prompt to a user; capturing, by a screen-associated camera of the smart device, facial image data and respiratory movement data without physical contact with the user; recording, by an integrated microphone of the smart device, voice data of the user; receiving, by an environmental sensing interface of the smart device, particulate matter information; applying, by one or more processors, a signal-quality gate to the captured data; extracting facial features from the facial image data; processing the respiratory movement data using Eulerian Video Magnification or video motion processing to extract respiratory features; extracting acoustic features from the voice data; generating environmental features from the particulate matter information; fusing the facial features, respiratory features, acoustic features, and environmental features by a DrAI analysis module; and outputting a preliminary non-diagnostic health assessment or real-time early warning alert through the smart device.

13. The method of claim 12, wherein extracting the facial features includes extracting complexion, expression, micro-expression, or eye-movement features.

14. The method of claim 12, wherein processing the respiratory movement data includes selecting a region of interest in video frames, applying temporal filtering, and generating a respiratory feature vector.

15. The method of claim 12, wherein extracting the acoustic features includes extracting pitch, energy, spectral, timing, pause, or speech-rate features from the recorded voice data.

16. The method of claim 12, further comprising aligning the facial features, respiratory features, acoustic features, and environmental features within a monitoring session time window before fusing the features.

17. The method of claim 12, further comprising displaying a repeat-capture prompt when the signal-quality gate indicates inadequate visual, acoustic, or environmental data.

18. The method of claim 12, wherein outputting the preliminary non-diagnostic health assessment or real-time early warning alert includes displaying the output on the smart device screen in real time.

19. The method of claim 12, wherein the preliminary non-diagnostic health assessment or real-time early warning alert is generated without requiring a body-contact sensor attached to the user.

20. A non-transitory computer-readable medium storing instructions that, when executed by one or more processors of a smart device having a screen-associated camera, microphone, and environmental sensing interface, cause the one or more processors to perform operations comprising capturing facial image data and respiratory movement data by the screen-associated camera, recording voice data by the microphone, receiving particulate matter information by the environmental sensing interface, applying a signal-quality gate, extracting facial features, extracting respiratory features using Eulerian Video Magnification or video motion processing, extracting acoustic features, generating environmental features, fusing the extracted features using a DrAI analysis module, and outputting a preliminary non-diagnostic health assessment or real-time early warning alert through the smart device.
